(19) United States
(12) Patent Application Publication
(10) Pub. No.: US 2017/0042298 A1
(43) Pub. Date: Feb. 16, 2017

(54) Gecko Jewelry

(71) Applicant: Ted Greg Lee Frumkin, Herzeliya (IL)

(72) Inventor: Ted Greg Lee Frumkin, Herzeliya (IL)

(21) Appl. No.: 15/234,500

(22) Filed: Aug. 11, 2016

Related U.S. Application Data
(60) Provisional application No. 62/203,930, filed on Aug. 12, 2015.

(51) Int. Cl. A44C 15/00; A44C 5/00; G04B 37/14; A44C 17/00; A61N 1/04; A61F 13/02; A44C 9/00; A44C 7/00

(52) U.S. Cl. CPC A44C 15/0005; A44C 9/00; A44C 15/005; A44C 5/00; A44C 7/00; A44C 17/00; A61N 1/0496; A61F 13/0256; G04B 37/14

(57) ABSTRACT

The present invention is directed towards jewelry products, connected with reusable stickiness (adhesive) to grab and release the body and different surfaces. The present invention also includes different purposes for the gecko jewelry such as a gecko diamond, wristwatch, phones, bandages, electrodes etc. While the gecko's “glue” holds the on the body strongly in one direction, one can easily pull the product out to the other direction. Due to the gecko jewelry, the shape of the product can be changed and not necessarily symmetric. The gecko jewelry can come as a complete new product or the gecko adhesive can be as an add-on a part for an existing jewelry. The present invention also include a jewelry that has reusable adhesive made out of carbon nanotubes mimicking a gecko foot. Realizing a jewelry grabbing on different parts of the body surface (or any other surfaces) by a special adhesive or any other glue product which doesn't leave residue, reusable, easy to use and doesn't wear off even in dusty or damp conditions. The present invention is directed to all jewelries for fashion or for medical use.

### PRIORITY DATA

The present application claims priority from U.S. provisional patent 62/203,930 filed on Aug. 12, 2015 by the inventor of the present application and is hereby incorporated by reference in its entirety.

### FIELD OF THE INVENTION

The present invention generally relates to product or items that are glued to the human body (will be referred as jewelry) such as watches, phones, necklaces, bracelets, earrings, diamonds and other gem stones etc. (will be referred as diamonds) or other body glued products such as bandages, elastic bandages, medical adhesive tapes, electrodes cloths and fabrics etc. (will be referred as bandages) for waring products on the body, and in particular, it concerns reusable adhesive on the jewelry and/or bandages and/or diamonds for gluing the product on the body.

### BACKGROUND OF THE INVENTION

Jewelry exists and is worn by humans since around 100,000 years ago and up to modern times. These days, in a world where new technologies are implemented in all areas of our life, and in particular with smart phones and watches, people increasingly use smart watches and even consider them as jewels. Up to now watches, necklaces, bracelets, earrings, diamonds and other gem stones etc. have a lock that holds them on the human body (either by a strap to hold your wristwatch on your hand or by a hole in the ear to where earrings with bracket, or a necklace to hold the diamond on your neck etc.). Some gemstones are glued to the human body conventional glue but it is used only ones don't stay glued for long and the glue needs to be replaced, when taken off it hurts (sometimes also tears off hair) and leave a mark on the body. As well in the medical industry, one subject is very common and yet wasn't implemented with the technologies that we use today. Bandages, elastic bandages, medical adhesive tapes, electrodes and other products that are glued to the human body. Up to now, Band-Aid or other body glued products, are disposable and used only once, when taken off it hurts (sometimes also tear off hair) and leaves a mark on the body (usually in a medical treatment the band-aid are attached and replaced several times). With this patent we propose a complete new way to wear body glued products such as jewelry, bandages etc., by using a material that has reusable stickiness that can hold on the body, easy to be attached and removed when needed and doesn't leave any residue.

Note, all materials that don't leave residue, have stickiness (by glue or other forces) that can grab products, and the stickiness doesn't wear off with water or even in dusty or damp conditions, the products can be easily freed and connected back again, can be used and will be referred as gecko “glue”.

Non-limiting exemplary embodiments of exemplary includes an adhesive imitating the geckos foot. The adhesive is made out of carbon nanotubes, it can hold on extremely strong in one direction, and can be easily freed on the other direction.

Geckos have the remarkable ability to cling to just about any surface, even something as smooth and slippery as glass. It's not due to suction, glue or Velcro-like bristles. Instead, geckos toe pads are covered with millions of tiny, flexible hairs. When a gecko plants its foot, the individual hairs come in extremely close contact with the surface is climbing on. That super-closeness makes it possible for atoms and molecules in the hairs to attract their counterpart atoms and molecules in the surface layer.

This attraction is called the Van Der Waals force (after the discoverer). The attraction is weak, but its effect is multiplied by the density and sheer number of hairs (or in the glue case the carbon nanotubes) on the gecko's toe pads. Each hair stalk, or seta, branches into hundreds of smaller, flattened tips called spatulae. The setae are able to shed dirt and to an extent, water, meaning a gecko's stickiness doesn't wear off even in dusty or damp conditions. To unstick its feet a gecko curls its toes upward (the other direction), peeling the setae away from the surface.

Referring to FIG. 1, inspired from the structure found on gecko feet, an optional embodiment synthesized carbon nanotube-based hierarchical hairy structure. Carbon nanotubes can be synthesized using chemical vapor deposition. Image of scanning electron microscope (SEM) of pillars of carbon nanotubes.

Referring to FIG. 2, non-limiting example patterns of pillared size ranging from 50-500 μm. The patterns act as setae and individual carbon nanotubes as spatula. These vertically aligned nanotubes can be transferred onto a polymeric substrate (the geometry would be referred as gecko tape). By optimizing the pattern size and the size and height of carbon nanotubes, very high shear forces can be achieved. For a non-limiting example 50 μm pattern size with 200 μm carbon nanotube length showed four times higher adhesion than natural gecko foot.

### SUMMARY

The present invention is directed to body glued products such as jewelry, bandages etc., connected with reusable stickiness to be placed or hanged on the human body. In addition to the adapted jewelry and medical bandages noted above, the present invention also includes the use of a carbon nanotube “glue” imitating the gecko's foot, for attaching the jewelry and bandages on the body. The “glue” imitating the gecko's foot can be hanged easily on the body in one direction while it can be taken off easily by pulling them to the other direction. As well the gecko's “glue” can be reused and it doesn't wear off even in dusty or damp conditions.

An additional advantage of the new inventive gecko jewelry is by the remarkable ability of the gecko imitating “glue” to stick on almost any surface, meaning one may hang the jewelry on different kind of surfaces. For example hang the diamonds on the wall at the bedroom for safe keep, or hang the cellular phone on the dashboard in the car. The gecko jewelry glued product can have different shapes and can be hanged sideways or upside down, depending on the location and shape of the product. This concept is applicable to all types of glued products that can be glued on a human body and/or on clothes.

An additional advantage of the new inventive gecko bandage is that no residue is left on the body hence not causing any injuries, leaving no marks and improving hygienics, the gecko bandages are reusable (meaning that one can take it off easily rub some lotion and put it back again). The gecko medical glued product can have different shapes and can be placed sideways or upside down, depending on the location and shape of the medical problem. This concept is applicable to all types of medical glued products.

As mentioned, our objective is to realize a new way to wear products such as jewelry, bandages etc. glued on the human body with a reusable adhesive unlike what is common today, products that stick with glue on a human body, that leave marks on the body, pull out hairs and can cause injuries. Rather by a special gecko imitating adhesive that can stick easily and can be taken off easily, reusable, does not leave any residue hence do not leave marks on the body. Note: curved cellular phones can be glued on the body as well.

In an exemplary, non-limiting embodiment the adhesive consists of the carbon nanotubes imitating the gecko's foot, can stick on the human body from the back (the side that sticks to the body) of the jewelry or the bandage, cellular phone etc. without leaving marks and injuries, easy to attach and take off, and reattach in a different location. To attach, take off and reattach, the gecko jewelry or bandage just needs a pull to the opposite direction, the gecko jewelry or bandage can be reusable.

The gecko jewelry will be made by a thin layer (the thickness will be determined by the size and weight of the product) based on the back of the jewelry, bandages etc. The gecko adhesive can be dispersed on all the back part of the jewelry, or the gecko's “glue” can be dispersed only on certain locations at the back of the jewelry for creating a good enough stickiness, thus saving gecko “glue” material.

Additional advantage is due to the possibility of gluing products on the human body, the products shape could change (like a curved phone) to be more suitable to the area they will be placed on. For example the watch can be “glued” to the back of the palm and not on the wrist, anywhere suitable for the owner, and a diamond can be “glued” on the forehead etc. As well the jewelry products can be glued on clothes made from any kind of fabric.

An additional advantage, the gecko jewelry can be hanged on different location for safe storage.

### BRIEF DESCRIPTION OF FIGURES

FIG. 1: pillars of carbon nanotubes mimicking the setae.

FIG. 2: individual carbon nanotubes as the spatula.

FIG. 3A: non-limiting example of a jewelry, profile view of a diamond.

FIG. 3B: non-limiting example of a jewelry, profile view of a diamond with gecko adhesive on the bottom.

FIG. 3C: non-limiting example of a jewelry, profile view of a diamond with gecko adhesive that is dispersed only on certain areas.

FIG. 4A: non-limiting example of a jewelry, front side of a wristwatch.

FIG. 4B: non-limiting example of a jewelry, back side of a wristwatch with gecko adhesive.

FIG. 4C: non-limiting example of a jewelry, back side of a wristwatch with gecko adhesive that is dispersed only on certain areas.

FIG. 5A: non-limiting example of a jewelry, front side of a cellular phone.

FIG. 5B: non-limiting example of a jewelry, back side of the cellular phone with gecko adhesive.

FIG. 5C: non-limiting example of a jewelry, back side of the cellular phone with gecko adhesive that is dispersed only on certain areas.

FIG. 6A: non-limiting example of bandages, front side of a bandage.

FIG. 6B: non-limiting example of bandages, back side of a bandage covered by gecko adhesive.

FIG. 6C: non-limiting example of bandages, back side of a bandage covered by gecko adhesive that is dispersed only on certain locations.

FIG. 7A: non-limiting example of bandages, front side of medical electrode.

FIG. 7B: non-limiting example of bandages, back side of a medical electrode covered by gecko adhesive.

FIG. 7C: non-limiting example of bandages, back side of a medical electrode covered by gecko adhesive that is dispersed only on certain locations.

### DETAILED DESCRIPTION

The principles and operation of the apparatus and method according to a present embodiment may be better understood with reference to the drawings and the accompanying description. A present invention is an apparatus for an adhesive of body-glued products. The apparatus facilitates gluing jewelry and other products on the body, using an innovative gecko adhesive. The gecko jewelry holds the surface of the product with reusable adhesive material that does not leave residue, in order to stick and unstick. The jewelries shapes with the gecko adhesive can change due to the possibility of the gecko adhesive to stick perpendicular and parallel to the ground (i.e. against gravity) and can be adjusted to the location the jewelry is directed for.

Current research in the field is focused on using conventional glues that are not reusable hard to take off and can cause injuries.

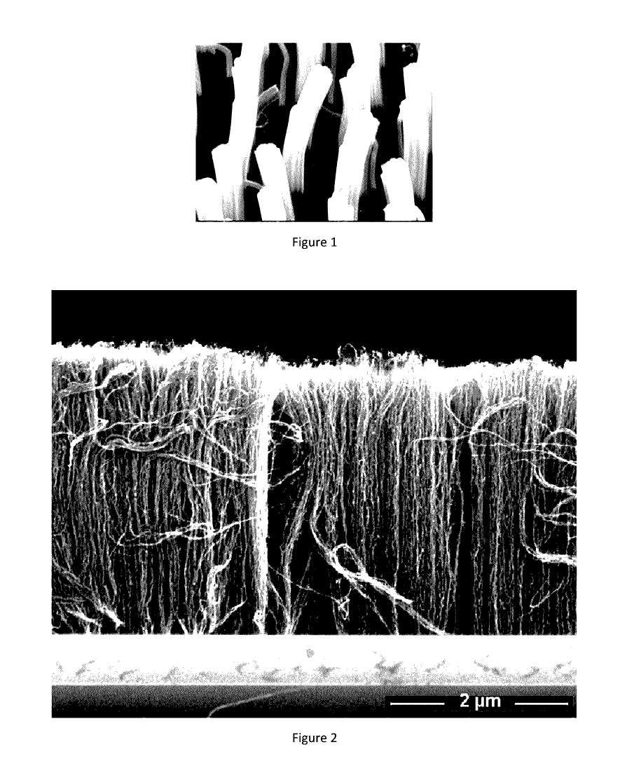

Refer to FIG. 1, non-limiting example of a top view SEM picture of pillars contain out of carbon nanotubes. The pillars act as the setae in order to create adhesive mimicking the gecko's foot. The pillars size and shape can be adjusted to a specific product in order to get the best adhesive.

Refer to FIG. 2, non-limiting example of a side view zoom of a pillar a cluster of carbon nanotubes, individual carbon nanotube can be seen. The individual carbon nanotubes act as the spatula in order to create adhesive mimicking the gecko's foot. The carbon nanotube size and height can be adjusted to a specific product demands.

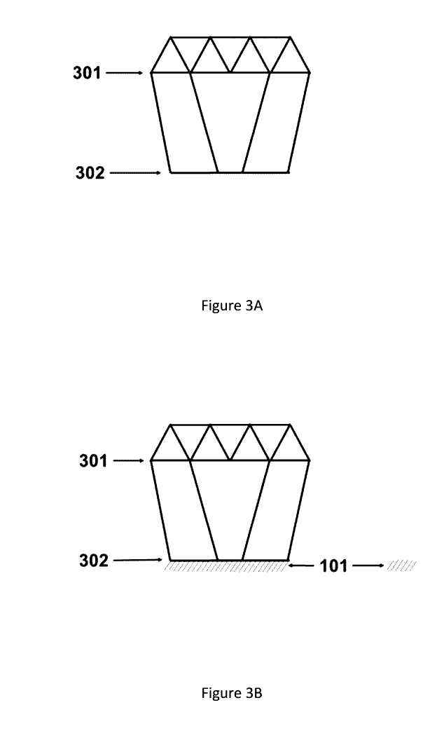

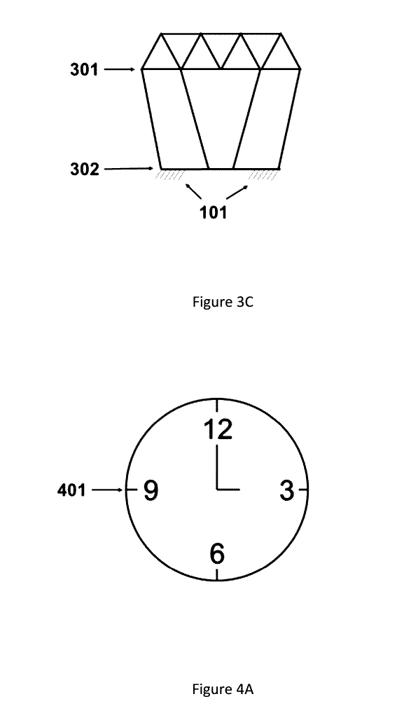

Refer to FIG. 3A, non-limiting example of a profile view of a diamond 301. Where the bottom of the diamond 302 is the location where it is connected or glued in order to keep the shiny part open to see. Note that in this case the diamond represent all kinds of jewelry.

Refer to FIG. 3B, non-limiting example of a profile view of a diamond 301. Where the bottom of the diamond 302 (back side that sticks to the surface of the body or any other surface) is fully covered with 101 gecko adhesive. The gecko adhesive can attach easily and can be removed easily from the body surface. Due to the gecko adhesive the jewelry can be stored (glued on different surfaces) for safe keeping after using glued on the location of our choosing, even on surfaces as smooth and slippery as glass, as well the gecko jewelry can connect to cloths or fabric etc. With the reusable ability of the gecko adhesive the jewelry can connect and disconnect to the body as many times needed without leaving any marks, residue or causing injuries.

Refer to FIG. 3C, non-limiting example of a profile view of a diamond 301. Where the bottom of the diamond 302 (back side that sticks to the surface of the body or any other surface) is covered with 101 gecko adhesive that is dispersed only on certain areas. Depending on the size and weight of the jewelry, the amount and quality of the carbon nanotubes and pillars can change, meaning that the gecko adhesive can be dispersed only on certain locations.

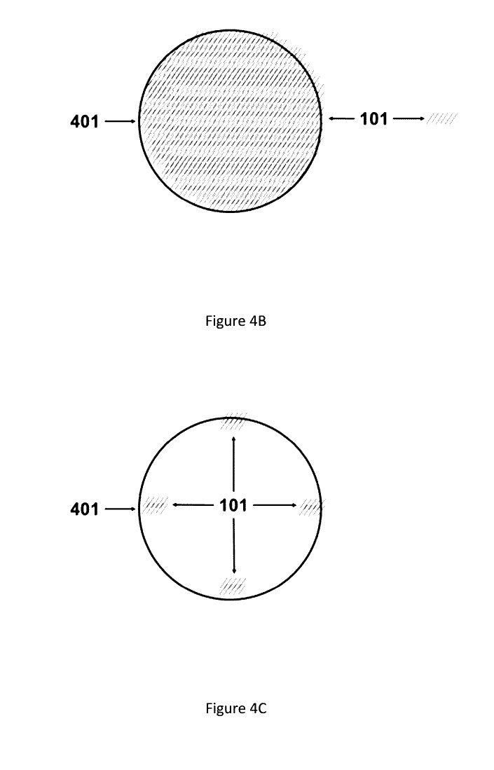

Refer to FIG. 4A, non-limiting example of a wristwatch 401 front side. One may see that the wristwatch doesn't have a wristband, due to the lack of wristband the shape of the watch don't have to be symmetrical thus the shape can change.

Refer to FIG. 4B, non-limiting example of a wristwatch 401 back side (that sticks on the surface of the body or any other surface). Where the back of the watch is fully covered with gecko adhesive 101. The gecko adhesive can attach easily and can be removed easily from the body surface. Due to the gecko adhesive the wristwatch can be stored (glued on different surfaces) for safe keeping after using glued on the location of our choosing, even on surfaces as smooth and slippery as glass, as well the gecko jewelry can connect to cloths or fabric etc. With the reusable ability of the gecko adhesive, the wristwatch can connect and disconnect to the body as many times needed without leaving any marks, residue or causing injuries.

Refer to FIG. 4C, non-limiting example of a wristwatch 401 back side (that sticks on the surface of the body or any other surface). Where the back of the watch is partially covered with gecko adhesive 101. Depending on the size and weight of the jewelry, the amount and quality of the carbon nanotubes and pillars can change, meaning that the gecko adhesive can be dispersed only on certain locations.

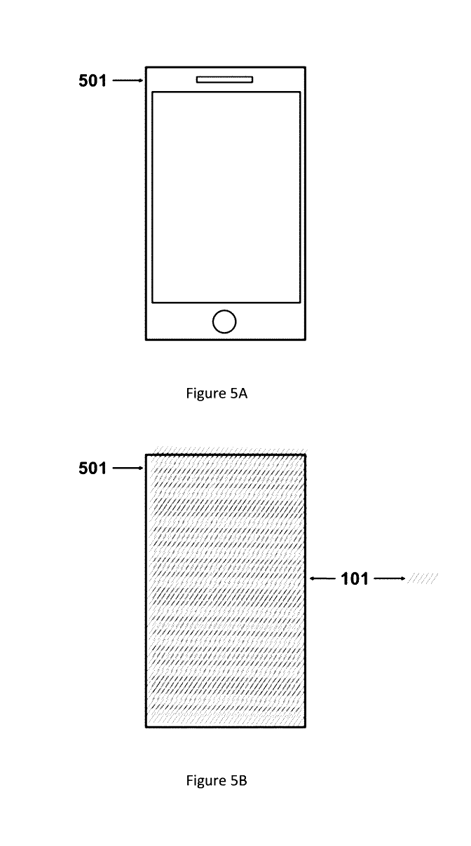

Refer to FIG. 5A, non-limiting example of a phone 501 front side. Companies are building new and flexible phones, up to the point where one can fold the phone like a paper. Flexible phones can be adjusted to be wear on arms, wrists etc. hence the shape of the new flexible phones can change.

Refer to FIG. 5B, non-limiting example of a phone 501 back side (that sticks on the surface of the body or any other surface). Where the back of the phone is fully covered with gecko adhesive 101, The gecko adhesive can attach easily and can be removed easily from the body surface. Due to the gecko adhesive the phone can be stored (glued on different surfaces) for safe keeping glued on the location of our choosing, even on surfaces as smooth and slippery as glass, as well the gecko jewelry can connect to cloths or fabric and the dashboard in the car etc. With the reusable ability of the gecko adhesive, the phone can connect and disconnect to the body or any other surfaces as many times needed without leaving any marks, residue or causing injuries.

Refer to FIG. 5C, non-limiting example of a phone 501 back side (that sticks on the surface of the body or any other surface). Where the back of the phone is partially covered with gecko adhesive 101. Depending on the size and weight of the phone, the amount and quality of the carbon nanotubes and pillars can change, meaning that the gecko adhesive can be dispersed only on certain locations.

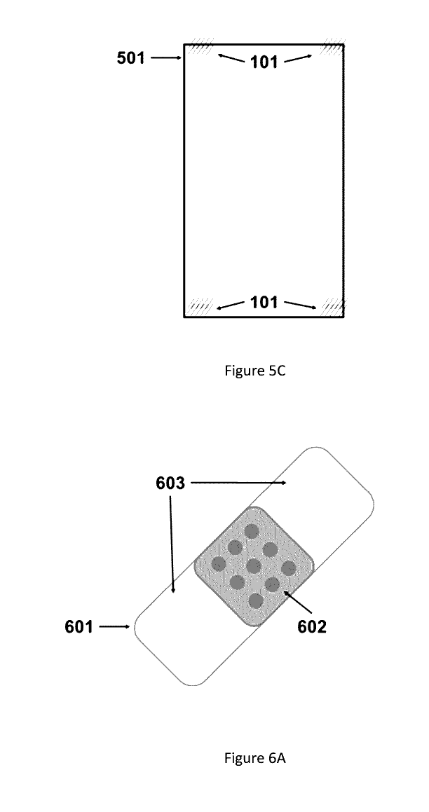

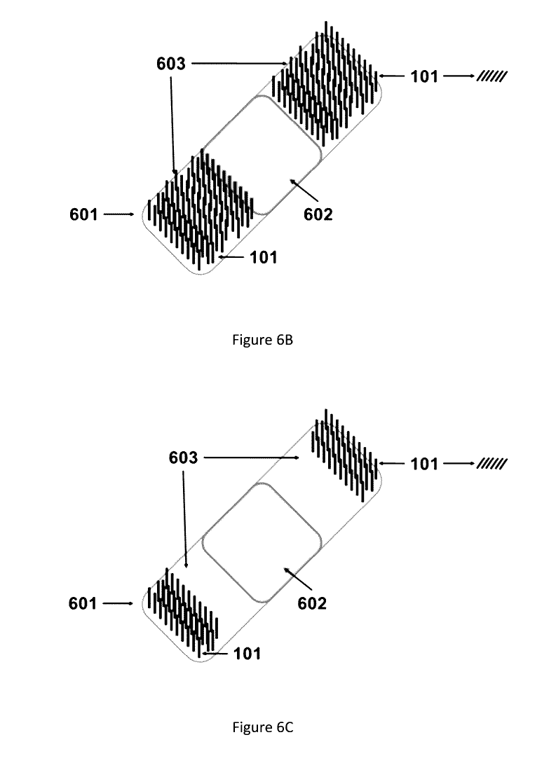

Refer to FIG. 6A, non-limiting example of a bandage 601 front side. Where 603 is the front part of the area that is glued to the body and 602 is the front part of the area that covers the wound. Note this bandage is used only ones and not reusable, ones the glue is open it cannot be restored.

Refer to FIG. 6B, non-limiting example of a bandage 601 back side (the side that sticks on the body) where the glue area 603 is covered with gecko adhesive 101. The gecko adhesive is covering the entire surface apart from 602 the area that covers the wound. The gecko adhesive can attach easily and can be removed easily from the body surface. With the reusable ability of the gecko adhesive, the bandage can connect and disconnect to the body as many times needed without leaving any marks, residue or causing injuries thus easy to clean and compress the wound. The size and shape of the gecko bandage can change depending on the treatment needed.

Refer to FIG. 6C, non-limiting example of a bandage 601 back side (the side that sticks on the body) where the glue area 603 is partially covered with gecko adhesive 101. The gecko adhesive is covering only parts of 603, on 602 the area that covers the wound there is no gecko adhesive. Depending on the size and shape of the bandage, the amount and quality of the carbon nanotubes and pillars can change, meaning that the gecko adhesive can be dispersed only on certain locations.

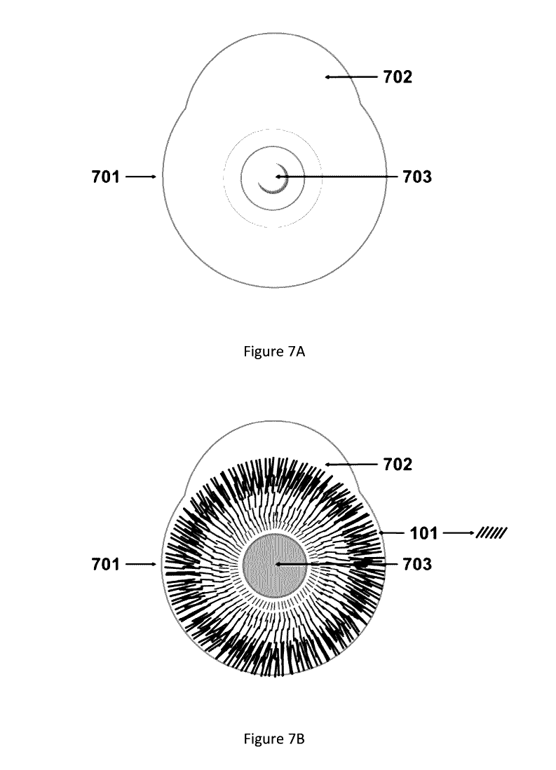

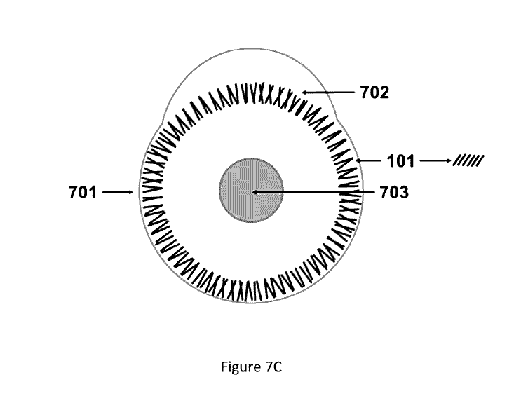

Refer to FIG. 7A, non-limiting example of an electrode 701 front side. Where 702 is the front part of the area that is glued to the body and 703 is the front part of the electrode that is connected to the machinery. Note this electrode is glued only ones and not reusable, ones the glue is open it cannot be restored.

Refer to FIG. 7B, non-limiting example of an electrode 701 back side (the side that sticks on the body) where the glue area 702 is covered with gecko adhesive 101. The gecko adhesive is covering the entire surface of 702. 703 is the back side of the electrode that touches the body (note that the electrode itself can be covered by the gecko adhesive making aria 702 unnecessary). The gecko adhesive can attach easily and can be removed easily from the body surface. With the reusable ability of the gecko adhesive, the electrode can connect and disconnect to the body as many times needed without leaving any marks, residue or causing injuries thus easy to clean. The size and shape of the gecko electrode can change depending on the treatment needed.

Refer to FIG. 7C, non-limiting example of an electrode 701 back side (the side that sticks on the body) where the glue area 702 is partially covered with gecko adhesive 101. The gecko adhesive is covering only parts of 702. 703 the area of the electrode there is no gecko adhesive (different way is with gecko adhesive on the electrode itself). Depending on the size and shape of the electrode, the amount and quality of the carbon nanotubes and pillars can change, meaning that the gecko adhesive can be dispersed only on certain locations.

A gecko jewelry has been shown to provide improved and easier way to glue products on the body compared to conventional techniques, and is a preferred implementation. Alternatively, the shape of the jewelry can change due to the lack of wristband or neckless etc. in order to stick the product on the body. Both different shapes and different locations to add the jewelry can be achieved.

The choices used to assist in the description of this embodiment should not detract from the validity and utility of the invention. It is foreseen that more general choices including, but not limited to materials, different shape size and configuration can be used, depending on the application.

The use of simplified calculations to assist in the description of this embodiment should not detract from the utility and basic advantages of the invention.

It should be noted that the above-described examples, numbers used, and exemplary calculations are to assist in the description of this embodiment. Inadvertent typographical and mathematical errors should not detract from the utility and basic advantages of the invention.

It will be appreciated that the above descriptions are intended only to serve as examples, and that many other embodiments are possible within the scope of the present invention as defined in the appended claims.

What is claimed:

1. Wearable article comprising:
An article body; and
a contact surface for attaching to a surface of a wear, wherein said contact surface include microstructure, nanostructure or picostructure based adhesive.

2. The article according to claim 1, wherein the article body may be selected from the group consisting of a bandage, an elastic bandage, a medical adhesive tape or electrode.

3. The article according to claim 1, wherein the body of the article is an article selected from the group consisting of jewelry, watch and phone.

4. The article according to claim 3, wherein the jewelry may be selected from the group consisting of a ring, a necklace, a bracelet, an earring, a diamond and another gem stone.

5. The article according to claim 3, wherein the contact surface have the ability to grab onto different surfaces.

6. The article according to claim 3, wherein the jewelry shape and purpose can be changed.
